(19) United States
(12) Patent Application Publication
(10) Pub. No.: US 2016/0175351 A1
(43) Pub. Date: Jun. 23, 2016

(54) CANCER TREATMENT

(71) Applicant: DUKE UNIVERSITY, Durham, NC (US)

(72) Inventors: Dennis J. Thiele, Chaple Hill, NC (US); Helena Öhrvik, Durham, NC (US)

(21) Appl. No.: 14/392,336

(22) PCT Filed: Jun. 24, 2014
(86) PCT No.: PCT/US2014/043802

§ 371 Date: Dec. 24, 2015

Related U.S. Application Data
(60) Provisional application No. 61/838,560, filed on Jun. 24, 2013.

(51) Int. Cl. A61K 33/24; A61K 45/06; C12Q 1/68

(52) U.S. Cl. CPC A61K 33/24; C12Q 1/6886; A61K 45/06; C12Q 2600/106; C12Q 2600/158

(57) ABSTRACT

Described herein are methods of inhibiting the proliferation of cancer cells and methods of treating cancer, as well as methods of predicting responsiveness of subjects to certain cancer treatments and methods of identifying subjects as candidates for certain cancer treatments.

### CROSS-REFERENCE TO RELATED APPLICATIONS

This application claims priority to U.S. Provisional Patent Application No. 61/838,560, filed on Jun. 24, 2013, the entire contents of which are hereby incorporated by reference.

### STATEMENT REGARDING FEDERALLY SPONSORED RESEARCH

This invention was made with government support under R01 DK074192 awarded by the National Institutes of Health (National Institute of Diabetes and Digestive and Kidney Diseases). The United States government has certain rights in the invention.

### BACKGROUND

Cisplatin is a platinum-based anti-cancer drug that is highly effective and currently in broad clinical use for the treatment of a number of cancers, including head, neck, ovarian, breast, pancreatic, testicular, melanoma, bladder, lung, sarcoma, squamous cell carcinoma, small cell lung cancer as well as others. Cisplatin and related platinum-based chemotherapeutic agents such as oxaliplatin, carboplatin, satraplatin, picoplatin and the like, are typically administered at high concentrations to enhance efficacy, but can have significant side-effects such as ototoxicity and nephrotoxicity. Furthermore, many cancer cells may develop resistance to platinum-based chemotherapeutics. Some forms of resistance may be intrinsic, wherein the cells or patients may be inherently resistant for reasons that are not well understood.

### SUMMARY

In one aspect, the disclosure provides a method of reducing the proliferation of a cancer cell, comprising contacting the cancer cell with a platinum-based chemotherapeutic and a cysteine protease inhibitor, such as a cathepsin inhibitor (e.g., a Cathespin L inhibitor).

In another aspect, the disclosure provides a method of treating cancer in a subject in need of treatment, comprising administering to the subject a platinum-based chemotherapeutic and a cysteine protease inhibitor, such as a cathepsin inhibitor (e.g., a Cathespin L inhibitor), in amounts effective to treat the cancer.

In another aspect, the disclosure provides a method of predicting responsiveness of a subject having cancer to treatment with a platinum-based chemotherapeutic agent, comprising:

providing a nucleic acid-containing sample obtained from the subject; and

detecting a Ctr1 nucleotide sequence selected from the group consisting of SEQ ID NO:1 and SEQ ID NO:2;

wherein the presence of SEQ ID NO:1 indicates that the subject is a responder to treatment with a platinum-based chemotherapeutic in the absence of a cysteine protease inhibitor, such as a cathepsin inhibitor (e.g., a Cathespin L inhibitor), and wherein the presence of SEQ ID NO:2 indicates that the subject is a non-responder to treatment with a platinum-based chemotherapeutic in the absence of a cysteine protease inhibitor, such as a cathepsin inhibitor (e.g., a Cathespin L inhibitor).

In another aspect, the disclosure provides a method of treating cancer in a subject in need of treatment, comprising:

providing a nucleic acid-containing sample obtained from the subject;

detecting a Ctr1 nucleotide sequence selected from the group consisting of SEQ ID NO:1 and SEQ ID NO:2; and

if the detecting step detects the presence of SEQ ID NO:2, the method further comprises administering to the subject a therapeutically effective amount of a platinum-based chemotherapeutic and a cysteine protease inhibitor, such as a cathepsin inhibitor (e.g., a Cathespin L inhibitor).

In another aspect, the disclosure provides a method of predicting responsiveness of a subject having cancer to copper chelation therapy, comprising:

providing a nucleic acid-containing sample obtained from the subject; and

detecting a Ctr1 nucleotide sequence selected from the group consisting of SEQ ID NO:1 and SEQ ID NO:2;

wherein the presence of SEQ ID NO:1 indicates that the subject is a responder to copper chelation therapy, and wherein the presence of SEQ ID NO:2 indicates that the subject is a non-responder to copper chelation therapy.

Other aspects and embodiments are encompassed by the disclosure and will become apparent in light of the following description and drawings.

### BRIEF DESCRIPTION OF THE DRAWINGS

FIG. 1 depicts a model for the cleavage of the Ctr1 ecto-domain. The Ctr1 copper/cisplatin transporter is shown in dark gray, with dotted lines pointing to the cisplatin-binding methionine residues. Cisplatin (or other platinum-based chemotherapeutic) is shown as gray balls. The Ctr2 protein, which forms a complex with Ctr1, is shown in light gray. Cathepsin L, a protease which cleaves off the cisplatin-binding ecto-domain of Ctr1, is shown in medium gray.

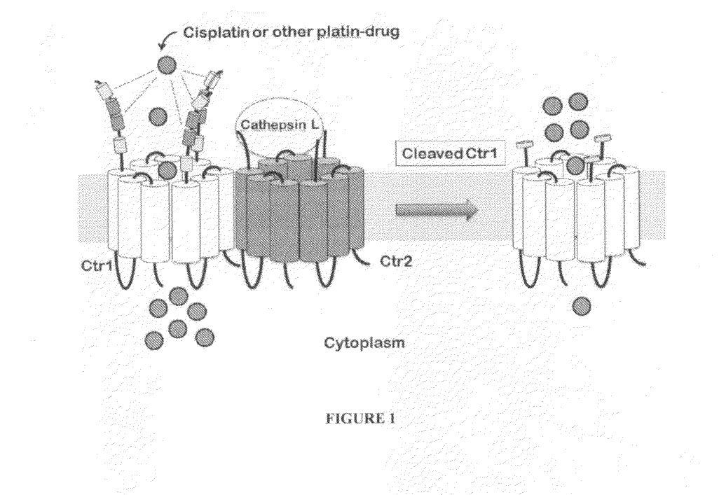

FIG. 2 is an immunoblot of protein extracts from mouse embryonic fibroblasts (MEFs) from wild-type (Ctr2^{+/+}), heterozygous (Ctr2^{+/-}) and knock out (Ctr2^{-/-}) cells, with an anti-Ctr1 antibody and an anti-tubulin antibody.

FIG. 3 illustrates two graphs of platinum (left) and copper (white) levels (in ng/mg protein) from wild-type MEFs (Ctr2^{+/+}, white bars) and in knock-out MEFs (Ctr2^{-/-}, gray bars).

FIG. 4 is an immunoblot of protein extracts from wild-type MEFs (lanes 1-4) and Cathepsin L^{-/-} MEFs (lanes 5-8) with an anti-Ctr1 antibody and an anti-tubulin antibody. Lanes 1 and 2 are duplicate samples from untreated wild type MEFs. Lanes 3 and 4 are duplicate samples from wild-type MEFs treated with the Cathepsin L inhibitor E64d. Lanes 5 and 6 are duplicate samples from untreated Cathepsin L^{-/-} MEFs. Lanes 7 and 8 are duplicate samples from Cathepsin L^{-/-} MEFs treated with the Cathepsin L inhibitor E64d.

FIG. 5 shows an immunoblot of protein extracts from wild-type MEFs (Ctr1) and from MEFs expressing Ctr1 with a Pro25Ala mutation (Ctr1^{P25A}), with an anti-Ctr1 antibody and an anti-actin antibody. Each of the above were either untreated (lanes 1 and 2), or treated with the Cathepsin L inhibitor Z-FY(tBu)-DMK (lanes 3 and 4). F=full-length, T=truncated.

FIG. 6 is a graph showing platinum accumulation in Ctr1 knockout MEFs (Ctr1^{-/-}), wild-type MEFs (Ctr1), or MEFs expressing Ctr1 with a Pro25Ala mutation (Ctr1^{P25A}). Each of the above were either untreated (1, 2 and 3), or treated with the Cathepsin L inhibitor Z-FY(tBu)-DMK (4, 5 and 6).

### DETAILED DESCRIPTION

The present disclosure is generally directed to methods of treating cancer and to methods of reducing the proliferation of cancer cells. The disclosure may also provide methods of predicting the responsiveness of subjects to certain cancer treatments; for example, the methods may involve identification of subjects as candidates for various cancer treatments.

A major uptake mechanism for the import of cisplatin in both yeast and mammalian cell is the Ctr1 copper importer (Ishida et al. (2002) Proc. Natl. Acad. Sci., USA 99: 14298-14302; Pope et al. (2012) Curr. Top. Membr. 69: 97-112; Kuo el al. (2012) Cancer Res. 72: 4616-4621). Ctr1 is an integral plasma membrane protein that is conserved from yeast to humans. Ctr1 has been demonstrated to constantly recycle from the cell surface (plasma membrane) to intracellular vesicles and back. Furthermore, in response to elevated levels of either copper or cisplatin, Ctr1 undergoes endocytosis from the plasma membrane to intracellular vesicles. Studies report that non-small cell lung cancer patients treated with platinum-based anti-cancer drugs have better survival rates when they express higher levels of Ctr1 than when they express lower levels of Ctr1 (Chen et al. (2012) Lung Cancer 75: 228-234). This is also true for patients with ovarian cancer where the observed survival rate is higher for patients with high Ctr1 expression treated with platinum-based chemotherapy, compared to patients with low Ctr1 expression (Lee et al. (2011) Gynecol. Oncol. 122: 361-365). By contrast, high Ctr2 expression associates with lower survival in the same patients, emphasizing the fact that both Ctr1 and Ctr2 expression will affect the uptake of platinum-based drugs and, consequently, have an important role for the patient's survival.

Previous studies have demonstrated that yeast cells or mouse embryonic fibroblasts that lack the Ctr1 gene are more resistant to cisplatin than the corresponding isogenic wild type cells, and accumulate much less cisplatin than the wild type cells (Ishida et al. (2002) Proc. Natl. Acad. Sci., USA 99: 14298-14302). Moreover, it appears that the mechanisms underlying copper transport and cisplatin transport by Ctr1 are mechanistically distinct (Sinani et al. (2007) J. Biol. Chem. 282: 26775-26785). Copper import appears to be accomplished by the movement of copper ions through an intra-membrane channel formed by homo-trimeric Ctr1. In contrast, studies suggest that cisplatin import is largely dependent on the binding of cisplatin to the methionine-rich clusters in the Ctr1 extracellular domain (ectodomain), followed by endocytosis of the Ctr1-cisplain complex into an endosomal compartment, which may release cisplatin by intracellular membrane fusion or other events (Ishida et al. (2002) Proc. Natl. Acad. Sci., USA 99: 14298-14302; Crider et al. (2010) Metallomics 2: 74-83). Moreover, a specific Ctr1 mutant that is defective for copper transport, is able to drive cellular cisplatin uptake. However, the methionine-rich regions of the Ctr1 ectodomain are dispensible for copper uptake, but required for cisplatin uptake (Sinani et al. (2007) J. Biol. Chem. 282: 26775-26785). Furthermore, it has been demonstrated that platinum chemotherapeutic agents directly coordinate to the methionine-rich motifs in the Ctr1 ectodomain (Crider et al. (2010) Metallomics 2:74-83). These observations demonstrate that the Ctr1 ectodomain is important for the import of cisplatin, and other platinum-based anti-cancer drugs, mediated by Ctr1.

The ectodomain of Ctr1, containing the methionine-rich regions, is variably cleaved in cultured rodent and human cells in endosomal compartments (Maryon et al. (2007) J Biol. Chem. 282:20376-20387). This cleavage is also observed in mouse tissues, but the precise cleavage sites were not identified, nor was the protease identified that carries out the cleavage of the Ctr1 ectodomain. More recently, the cleavage sites in Ctr1 were identified, and it was demonstrated that cleavage removes most of the methionine residues that are critical for enhancing platinum-based chemotherapeutic uptake as described above (Ohrvik et al. (2013) Proc. Natl. Acad. Sci. 110(46):E4279-4288). Additionally, a recombinant truncated form of Ctr1 that was expressed in cultured cells was shown to drive copper import with approximately 50% the efficiency of the corresponding wild type human Ctr1 (Maryon et al. (2007) J. Biol. Chem. 282:20376-20387).

The Ctr2 protein is structurally related to Ctr1 and is encoded by a linked gene in both the mouse and the human genome. Recent studies suggest that Ctr2 functions as a low-affinity Cu⁺ importer, a lysosomal Cu⁺ exporter, or as a regulator of cellular macropinocytosis (ven den Berghe et al. (2007) Biochem. J. 407(1):49-59); Blair et al. (2011) Mol. Pharmacol. 79(10): 157-166; Bertinato et al. (2008) Biochem. J. 409(3):731-740). More recent studies in mouse knock out animals and in mouse embryonic fibroblasts derived from Ctr2 knock-out mice demonstrate that Ctr2 is required for efficient cleavage of the Ctr1 ecto-domain (Öhrvik et al. (2013) Proc. Natl. Acad. Sci. 110(46):E4279-4288).

In this disclosure, two main factors are identified that are involved in cleavage of the mammalian Ctr1 ectodomain. One is a cysteine protease, which plays a major role in Ctr1 ectodomain cleavage. Such proteases involve the Cathepsin proteases, such as Cathepsin L, B, C, F, H, K, V, O, S and W. One such protease that has been identified is the lysosomal protease, Cathepsin L. Another is the Ctr2 protein, which stimulates Ctr1 cleavage. For example, it is demonstrated that Ctr2 interacts with Ctr1 in vivo and that Ctr2 knockout mice show increased levels of total copper in several tissues. Mice and mouse embryonic fibroblasts lacking Ctr2 accumulate copper in endosomal compartments and have lower levels of the truncated form of Ctr1 lacking the metal-binding ecto-domain. Whereas truncation of the Ctr1 ecto-domain reduces Cu⁺ import at the plasma membrane, truncated Ctr1 stimulates the mobilization of Cu⁺ from endosomal compartments. In view of these discoveries, the present disclosure is aimed at reducing cleavage of the mammalian Ctr1 ectodomain, in order to increase cellular uptake of cisplatin and other platinum-based chemotherapeutics. For example, inhibition of Cathepsin L and/or inhibition of the interaction between Ctr1 and Ctr2 may result in increased levels of Ctr1 protein that includes its cisplatin-binding ectodomain, which may result in increased cellular uptake of cisplatin and other platinum-based chemotherapeutics.

The disclosure also relates to the discovery of a single nucleotide polymorphism (SNP) in the Ctr1 gene that alters Proline-25 to an Alanine. When a gene encoding Ctr1 with the Pro25Ala mutation was expressed in mouse embryonic fibroblasts, the Ctr1 protein was present almost exclusively in a form in which the ectodomain is cleaved, compared to expression of wild-type Ctr1 which is present largely as the full-length glycosylated form. Therefore, identification of individuals with this SNP may serve as a diagnostic tool to predict responsiveness to treatment with a platinum-based chemotherapeutic, and to predict responsiveness to copper chelation therapy.

### 1. DEFINITIONS

“Administration” or “administering,” as used herein, refers to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants.

“Co-administered,” as used herein, refers to simultaneous or sequential administration of multiple compounds or agents. A first compound or agent may be administered before, concurrently with, or after administration of a second compound or agent. The first compound or agent and the second compound or agent may be simultaneously or sequentially administered on the same day, or may be sequentially administered within 1 day, 2 days, 3 days, 4 days, 5 days, 6 days, 1 week, 2 weeks, 3 weeks or one month of each other. Suitably, compounds or agents are co-administered during the period in which each of the compounds or agents are exerting at least some physiological effect and/or has remaining efficacy.

“Contacting,” as used herein as in “contacting a cell,” refers to contacting a cell directly or indirectly in vitro, ex vivo, or in vivo (i.e. within a subject, such as a mammal, including humans, mice, rats, rabbits, cats, and dogs). Contacting a cell, which also includes “reacting” a cell, can occur as a result of administration to a subject. Contacting encompasses administration to a cell, tissue, mammal, subject, patient, or human. Further, contacting a cell includes adding an agent to a cell culture. Other suitable methods may include introducing or administering an agent to a cell, tissue, mammal, subject, or patient using appropriate procedures and routes of administration as defined herein.

“Cathepsin L” as used herein refers to a lysosomal cysteine proteinase that plays a role in intracellular protein catabolism. This proteinase may also be referred to as “Cathepsin L1”. As used herein the term Cathepsin L encompasses any ortholog, variant, or functional fragment thereof. Multiple alternatively spliced transcript variants have been found for the gene CTSL1 which encodes the Cathepsin L1 protein; these include the sequences described in NCBI Reference Sequence Nos. NM_001912.1, NM_001912.2, NM_001912.3 and NM_001912.4. The Cathepsin L protein may include, for example, the sequence described in NCBI Reference Sequence Nos. NP_001903.1 and NP_666023.1.

“Ctr1” refers to a membrane associated, homotrimeric protein that transports reduced copper (Cu(I)) in to cells. As used herein, the term Ctr1 encompasses any ortholog, variant, or functional fragment thereof. Ctr1 can include, for example, the sequence described in NCBI Reference Sequence No. NP_001850.

“Ctr2” refers to a membrane associated, oligomeric protein that plays a role in regulating copper uptake in to cells along with Ctr1. As used herein, the term Ctr2 encompasses any ortholog, variant, or functional fragment thereof. Ctr2 can include, for example, the sequence described in NCBI Reference Sequence No. NP_001851.1.

“Effective amount,” as used herein, refers to a dosage of compounds or compositions effective for eliciting a desired effect. This term as used herein may also refer to an amount effective at bringing about a desired in vivo effect in an animal, mammal, or human, such as reducing proliferation of a cancer cell or treating cancer.

“Pharmaceutically acceptable,” as used herein, pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

“Reducing proliferation of a cell,” as used herein, refers to reducing, inhibiting, or preventing the survival, growth, or differentiation of a cell, including killing a cell. A cell can be derived from any organism or tissue type and includes, for example, a cancer cell (e.g., neoplastic cells, tumor cells, and the like).

The term “responder” refer to a subject or group of subjects having cancer, who show a clinically significant improvement when treated with a platinum-based chemotherapeutic. Conversely, a “non-responder” refers to a subject or group of subjects having cancer, who do not show a clinically significant improvement when treated with a platinum-based chemotherapeutic.

As used herein, the term “subject” is intended to include human and non-human animals. In embodiments, the subject is a human. Exemplary human subjects include a human patient having a disorder, e.g., cancer. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

“Susceptibility,” as used herein regarding a cancer cell, refers to the degree to which a cancer cell is affected by a chemotherapeutic agent. The cancer cell may not be affected at all, it may have its growth or proliferation slowed or halted without its being killed, or it may be killed. Susceptibility also refers to the degree a population of cancer cells, such as a tumor, is affected by a chemotherapeutic agent. “Increasing the susceptibility” of a cancer cell to a chemotherapeutic following contact or treatment with an agent, e.g., an inhibitor of an enzyme or an inhibitor of a protein-protein interaction, indicates that the cell is more affected by the chemotherapeutic agent than a corresponding cancer cell that has not been exposed to the agent.

As used herein, the term “treat” or “treating” a subject having a disorder refers to administering a regimen to the subject, e.g., the administration of a platinum-based therapeutic and/or another agent, such that at least one symptom of the disorder is healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

### 2. METHODS OF REDUCING THE PROLIFERATION OF CANCER CELLS AND TREATING CANCER

Disclosed herein are methods for reducing proliferation of a cancer cell, as well as methods for treating cancer in a subject in need of treatment. In some embodiments, the method comprises contacting a cancer cell with a platinum-based chemotherapeutic and a cysteine protease inhibitor, such as a Cathepsin L inhibitor. In other embodiments, the method comprises contacting a cancer cell with a platinum-based chemotherapeutic and an inhibitor of an interaction between Ctr1 and Ctr2.

a. Platinum-Based Chemotherapeutics

In each of the above-described embodiments, the method comprises contacting a cancer cell with a platinum-based chemotherapeutic agent. Such agents are among the most important groups of chemotherapeutic compounds currently in use, and are typified by cisplatin [cis-diamminedichloroplatinum(II)]. These agents, used alone or as a part of combination chemotherapy regimens, have been shown to be curative for testicular and ovarian cancers and may be beneficial for the treatment of lung cancer (e.g., small cell lung cancer), bladder cancer, head and neck cancers, breast cancer, pancreatic cancer, melanoma, sarcomas, and squamous cell carcinoma among many others.

DNA damage is believed to be a major determinant of cytotoxicity for platinum-based chemotherapeutics, though these drugs also may induce other types of cellular damage. Cisplatin is known to form adducts with DNA and to induce interstrand crosslinks. Adduct formation, through an as yet unknown signaling mechanism, is believed to activate some presently unknown cellular enzymes involved in programmed cell death (apoptosis), the process which is believed to be ultimately responsible for cisplatin cytotoxicity.

Platinum-based chemotherapeutics that may be used in the methods include, without limitation: cisplatin, carboplatin, oxaliplatin, iproplatin, tetraplatin, lobaplatin, dicycloplatin (DCP), PLD-147, JM118, JM335, satraplatin and picoplatin. Platinum-based chemotherapeutic agents also include platinum complexes disclosed in EP 0147926, U.S. Pat. No. 5,072,011, U.S. Pat. No. 5,244,919, U.S. Pat. No. 5,519,155, U.S. Pat. No. 6,503,943, U.S. Pat. No. 6,350,737, and WO 01/064696. These compounds are believed to act by the same or very similar mechanisms, so that conclusions drawn from studies of cisplatin sensitivity and resistance are expected to be valid for other platinum-containing drugs.

Embodiments of the methods described herein provide platinum coordination complexes wherein platinum is in the Pt(II) oxidation state. Some embodiments provide platinum coordination complexes having a square planar geometry with respect to the platinum atom.

b. Cysteine Protease Inhibitors

In some embodiments, the methods comprise contacting a cancer cell with a cysteine protease inhibitor, such as an inhibitor of Cathepsin L, B, C, F, H, K, V, O, S or W. Exemplary cysteine protease inhibitors include Cathepsin L inhibitors, Cathepsin B inhibitors, and Cathepsin H inhibitors. Particular cysteine protease inhibitors include Cathepsin L inhibitors. A Cathepsin L inhibitor may be any compound capable of reducing or eliminating the activity of Cathepsin L, such as a small molecule or an antibody. The Cathepsin L inhibitor may further comprise a small interfering RNA (siRNA) capable of interfering with the expression of Cathepsin L.

Certain small molecule inhibitors of Cathepsin L are known. These include, for example, the following:

Z-FF-FMK, also known as Cbz-Phe-Phe-fluoromethylketone or “Cathepsin L Inhibitor I”, having the chemical name benzyl (1-((4-fluoro-3-oxo-1-phenylbutan-2-yl)amino)-1-oxo-3-phenylpropan-2-yl)carbamate, CAS No. 108005-94-3;

Z-FY-CHO, also known as Cbz-Phe-Tyr-CHO or “Cathepsin L Inhibitor II”, having the chemical name benzyl (1-((1-(4-hydroxyphenyl)-3-oxopropan-2-yl)amino)-1-oxo-3-phenylpropan-2-yl)carbamate, CAS No. 167498-29-5;

Z-FY(tBu)-DMK, also known as Cbz-Phe-Tyr(tBu)-diazomethylketone or “Cathepsin L Inhibitor Ill”, having the chemical name benzyl (1-((1-(4-(tert-butoxy)phenyl)-4-diazo-3-oxobutan-2-yl)amino)-1-oxo-3-phenylpropan-2-yl)carbamate;

E-64, also known as trans-Epoxysuccinyl-L-leucylamido(4-guanidino)butane, L-trans-3-Carboxyoxiran-2-carbonyl-L-leucylagmatine, or N-(trans-Epoxysuccinyl)-L-leucine 4-guanidinobutylamide, CAS No. 66701-25-5;

E-64C, also known as (2S,3S)-trans-Epoxysuccinyl-L-lcucylamido-3-methylbutane, CAS No. 76684-89-4; and

E-64D, also known as (2S,3S)-trans-Epoxysuccinyl-L-leucylamido-3-methylbutane ethyl ester, CAS No. 88321-09-9.

Other Cathepsin L inhibitors are known, as disclosed, for example, at http://www.scbt.com/chemicals-table-cathepsin_l_inhibitors.html.

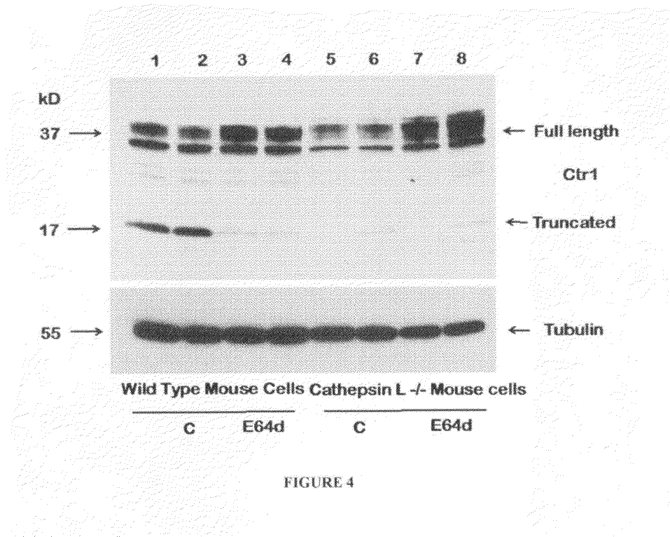

Certain compounds described herein as “Cathepsin L inhibitors” may inhibit not only Cathepsin L, but may also inhibit other cysteine proteases, such as other cathepsins. For example, E-64 is known to inhibit cathepsins B, H and L, as well as other cysteine proteases including calpain, papain and others. See, e.g., McGowan et al. (1989) Biochem. Biophys. Res. Commun. 158:432-435; Barrett et al. (1982) J. Biochem. 201:189-198. As will be described further in the Examples, as shown in FIG. 4, there appears to be an increase full-length Ctr1 in Cathepsin L −/− cells when treated with the Cathepsin L inhibitor E-64D. Therefore, E-64D may not only be inhibiting Cathepsin L, but may also be inhibiting another protease that may also be involved in Ctr1 ectodomain cleavage.

c. Inhibitors of Ctr1-Ctr2 Interaction

In some embodiments, the methods comprise contacting a cancer cell with a compound that inhibits an interaction between Ctr1 and Ctr2, generally referred to herein as a “Ctr1/Ctr2 inhibitor”. The compound may be any compound capable of reducing or eliminating the interaction between Ctr1 and Ctr2, such as a small molecule or an antibody. The Ctr1/Ctr2 inhibitor may further comprise a small interfering RNA (siRNA) capable of interfering with the expression of Ctr2.

d. Copper Chelation Therapy

Certain embodiments relate to methods of predicting responsiveness of a patient having cancer to copper chelation therapy.

Copper chelators for use in copper chelation therapy may include without limitation: penicillamine (Cuprimine®, Depen®), trientine hydrochloride (also known as triethylenetetramine hydrochloride, or Syprine®), dimercaprol, diethyldithiocarbamate (e.g., sodium diethyldithiocarbamate), bathocuproine sulfonate, and tetrathiomolybdate (e.g., ammonium tetrathiomolybdate).

e. Formulations

While compounds such as platinum-based chemotherapcutics, cysteine protease inhibitors such as Cathepsin L inhibitors, and Ctr1/Ctr2 inhibitors may be administered alone in the various methods described herein, they may also be presented as one or more pharmaceutical compositions (e.g., formulations). In each composition the compounds may be formulated with one or more pharmaceutically acceptable carriers, adjuvants, excipients, diluents, fillers, buffers, stabilizers, preservatives, lubricants, or other materials well known to those skilled in the art and optionally other therapeutic or prophylactic agents.

Accordingly, the methods described herein include administration of one or more pharmaceutical compositions, as discussed herein, in which a compound such as a platinum-based chemotherapeutic, cysteine protease inhibitor such as a Cathepsin L inhibitor, and/or Ctr1/Ctr2 inhibitor is admixed together with one or more pharmaceutically acceptable carriers, excipients, buffers, adjuvants, stabilizers, or other materials, as described herein. Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods known in the art of pharmacy. Such methods include the step of bringing into association the active compound(s) with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g. by injection, including cutaneous, subcutaneous, intramuscular, intravenous and intradermal), include aqueous and nonaqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include lozenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichoro-tetrafluoroethane, carbon dioxide, or other suitable gases. Further formulations suitable for inhalation include those presented as a nebulizer.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

f. Dosages

It will be appreciated that appropriate dosages of the active compounds, and compositions comprising the active compounds, can vary from patient to patient.

Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of an active compound is in the range of about 100 μg to about 250 mg per kilogram body weight of the subject per day.

For example, a suitable dose of a platinum-based therapeutic may be a standard dose. For example, a standard dosage of cisplatin for the treatment of testicular cancer is 20 mg/m² IV daily for 5 consecutive days every 3 weeks for 3 or 4 courses of therapy. A standard dosage of cisplatin for the treatment of advanced ovarian carcinoma is 30-120 mg/m² IV once every 3-4 weeks (e.g., 50-100 mg/m² IV once every 3 weeks, e.g., 100 mg/m² IV once every 4 weeks) when cisplatin is used as a single agent; 75 mg/m² IV once every 3 weeks in combination therapy with paclitaxel; or 50-100 mg/m² IV once every 3-4 weeks when used in combination with cyclophosphamide. For the treatment of advanced bladder cancer, a standard dosage of cisplatin is 50-70 mg/m² IV once every 3-4 weeks. A standard dosage for the treatment of recurrent or advanced head and neck cancer is 80-120 mg/m² IV once every 3 weeks or 50 mg/m² IV on the first and eighth days of every 4 weeks, when cisplatin is used as a single agent; when used in combination chemotherapy regimens, a standard dose is 50-120 mg/m² IV, with the frequency of administration depending on the specific regimen employed. A standard dosage of cisplatin for the treatment of cervical cancer, e.g., invasive cervical cancer, is 40-75 mg/m² have been given concurrently with radiation therapy, in weekly or daily infusions of cisplatin; when used in combination chemotherapy regimens (e.g., cisplatin and fluorouracil) for the treatment of invasive cervical cancer, cisplatin 50-75 mg/m² has been administered IV concurrently with radiation therapy. For the treatment of metastatic or recurrent cervical carcinoma, a standard dosage of cisplatin used alone or in combination therapy is 50 mg/m² IV once every 3 weeks up to a maximum of 6 courses. For the treatment of non-small cell lung carcinoma, a standard dosage of cisplatin in combination therapy is 75-100 mg/m² IV once every 3-4 weeks, depending on the specific regimen used. For the treatment of advanced esophageal cancer, a standard dosage of cisplatin 50-120 mg/m² IV once every 3-4 weeks; in combination chemotherapy regimens, a standard dosage cisplatin is 75-100 mg/m² IV once every 3-4 weeks.

A standard dosage of oxaliplatin for the treatment of advanced colorectal cancer is 85 mg/m² IV infusion and leucovorin 200 mg/m² IV infusion in dextrose 5% in water, both given over 120 min at the same time in separate bags using a Y-line, followed by 5-fluorouracil 400 mg/m² IV bolus given over 2 to 4 min, followed by 5-fluorouracil 600 mg/m² IV infusion in dextrose 5% in water 500 mL (recommended) as a 22-h continuous infusion.

A standard dosage of carboplatin for the treatment of ovarian cancer is 360 mg/m² by intravenous injection on day 1 every 4 weeks when used as a single agent; when used in combination with cyclophosphamide, a standard dosage of carboplatin 300 mg/m² by intravenous injection on day 1 every four weeks for six cycles. A standard dosage of carboplatin for the treatment of cervical cancer, in combination with other chemotherapeutic agents as a part of the BIC regimen, is 200 mg/m² IV on day 1; the cycle is repeated every 21 days.

In the methods described herein, contacting a cancer cell with a cysteine protease inhibitor such as a Cathepsin L inhibitor, or a Ctr1/Ctr2 inhibitor may lead to increased expression of a full-length Ctr1 protein and increased uptake of a platinum-based chemotherapeutic. Accordingly, dosages of platinum-based therapeutics that are lower than standard dosages may be effective in the methods described herein.

g. Cancer

The methods described herein can be used with any cancer cell or in a subject having any type of cancer, for example those described by the National Cancer Institute. The cancer can be a carcinoma, a sarcoma, a myeloma, a leukemia, a lymphoma or a mixed type. Exemplary cancers described by the National Cancer Institute include but are not limited to:

Digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer;

Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;

Eye cancers such as intraocular melanoma; and retinoblastoma;

Musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;

Breast cancer such as breast cancer including childhood and male breast cancer and breast cancer in pregnancy;

Neurologic cancers such as childhood brain stemglioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor;

Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor; Germ cell cancers such as childhood extracranial germ cell tumor; extragonadal germ cell tumor; ovarian germ cell tumor;

Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;

Hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as adult malignant mesothelioma; childhood malignant mesothelioma; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;

Skin cancers such as Kaposi's sarcoma; Merkel cell carcinoma; melanoma; and childhood skin cancer;

AIDS-related malignancies;

Other childhood cancers, unusual cancers of childhood and cancers of unknown primary site;

and metastases of the aforementioned cancers can also be treated or prevented in accordance with the methods described herein.

The methods described herein may be suited for bladder, testicular, ovarian, head and neck, cervical, lung (e.g., small cell lung), mesothelioma, esophageal, melanoma, brain tumor, neuroblastoma, colorectal, Wilms' tumor, retinoblastoma, breast, endometrial, adrenocortical, anal, biliary tract, carcinoid tumors, choriocarcinoma, gastric, liver cancer, non-Hodgkin's lymphoma, osteosarcoma, soft-tissue sarcomas, penile, malignant thymoma, anaplastic thyroid cancer, rhabdoid tumor of the kidney, advanced medullary thyroid cancer, carcinoid, mesothelioma, bone, gliomas, squamous cell carcinoma, pancratic or prostate cancers.

In embodiments, the methods may be used for bladder cancer (e.g., muscle-invasive bladder carcinoma, advanced or metastatic bladder carcinoma), testicular cancer (e.g., nonseminomatous testicular carcinoma, disseminated seminoma testis or extragonadal germ-cell tumors), ovarian cancer (e.g., ovarian epithelial cancer or ovarian germ-cell tumors), head and neck cancer (e.g., squamous cell carcinoma), breast cancer, pancreatic cancer, sarcomas, cervical cancer (e.g., invasive, metastatic or recurrent cervical cancer), lung cancer (e.g., small cell lung cancer or non-small cell lung cancer), Wilms' tumor, brain tumors (e.g., gliomas, medulloblastoma or germ cell tumors), neuroblastoma, retinoblastoma, mesothelioma (e.g., malignant pleural mesothelioma), esophageal cancer (e.g., localized or advanced esophageal cancer), melanoma, and colorectal cancer.

In embodiments, the methods may be suited for a cancer that is resistant to treatment with a platinum-based chemotherapeutic such as cisplatin (i.e. a cisplatin-resistant cancer).

h. Cancer Combination Therapy

Methods described herein may be used in further combination with other known therapies. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The platinum-based chemotherapeutic, the cysteine protease inhibitor such as a Cathepsin L inhibitor, and/or Ctr1/Ctr2 inhibitor, and the additional therapeutic agent can be administered simultaneously, in the same or in separate compositions, or sequentially.

In some embodiments, the platinum-based chemotherapeutic and the cysteine protease inhibitor such as a Cathepsin L inhibitor and/or Ctr1/Ctr2 inhibitor are administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various therapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the platinum-based chemotherapeutic and the cysteine protease inhibitor such as a Cathepsin L inhibitor and/or Ctr1/Ctr2 inhibitor are administered with at least one additional therapeutic agent, such as a chemotherapeutic agent. In certain embodiments, the platinum-based chemotherapeutic and the cysteine protease inhibitor such as a Cathepsin L inhibitor and/or Ctr1/Ctr2 inhibitor are administered in combination with one or more additional chemotherapeutic agents, e.g., with one or more chemotherapeutic agents described herein.

In some embodiments, the platinum-based chemotherapeutic and the cysteine protease inhibitor such as a Cathepsin L inhibitor and/or Ctr1/Ctr2 inhibitor are administered in combination with a chemotherapeutic agent. Exemplary classes of chemotherapeutic agents include, e.g., the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Endoxan®, Procytox®, Revimmune™), ifosfamide (Mitoxana®), melphalan (Alkeran®), Chlorambucil (Leukeran®), pipobroman (Amedel®, Vercytc®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

additional platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101).

taxanes: paclitaxel (Taxol®), docetaxel (Taxotere®), larotaxel, cabazitaxel.

epothilones: ixabepilone, epothilone B, epothilone D, BMS310705, dehydelone, ZK-Epothilone (ZK-EPO).

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

interferons (e.g., IFN-alpha (Alferon®, Roferon-A®) Intron®-A) or IFN-gamma (Actimmune®))

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected fromgcldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®) Keoxifene®) and raloxifene hydrochloride.

anti-hypercalcaemia agents which include without limitation gallium (HI) nitrate hydrate (Ganite@) and pamidronate disodium (Aredia®).

apoptosis inducers which include without limitation ethanol, 2-[[3-(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-pipe-razinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[1S)-3-fluoro-2-oxo-1-(2-phenylethyl)propyl]amino]-2-oxo-1-(phenylmethy-1)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation A-hydroxyfarnesylphosphonic acid, butanoic acid, 2-[(2S)-2-[[(2S,3S)-2-[[(2R)-2-amino-3-mercaptopropyl]amino]-3-methylpent-yl]oxy]-1-oxo-3-phenylpropyl]amino-1-4-(methylsulfonyl)-1-methylethylestcr (2S)-(9Cl), and manumycin A.

Flk-1 kinase inhibitors which include without limitation 2-propenamide, 2-cyano-3-[4-hydroxy-3,5-bis(l-methylethyl)phenyl]-N-(3-phenylpropyl)-(2E-)-(9Cl).

glycogen synthase kinase-3 (GSK3) inhibitors which include without limitation indirubin-3′-monooxime.

histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

imidazotetrazinones which include without limitation temozolomide (Methazolastone®, Temodar® and its derivatives (e.g., as disclosed generically and specifically in U.S. Pat. No. 5,260,291) and Mitozolomide.

insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hy-droxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9,1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No. 0 564 409 and PCT Publication No. WO 99/03854.

phosphatidylinositol 3-kinase inhibitors which include without limitation wortmannin, and quercetin dihydrate.

phosphatase inhibitors which include without limitation cantharidic acid, cantharidin, and L-leucinamide.

protein phosphatase inhibitors which include without limitation cantharidic acid, cantharidin, L-P-bromotetramisole oxalate, 2(5H)-furanonc, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-1-[[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

polyamine synthesis inhibitors which include without limitation DMFO.

proteasome inhibitors which include, without limitation aclacinomycin A, gliotoxin and bortezomib (Velcade®).

PTP1B inhibitors which include without limitation L-leucinamide. protein tyrosine kinase inhibitors which include, without limitation tyrphostin Ag 216, tyrphostin Ag 1288, tyrphostin Ag 1295, geldanamycin, genistein and 7H-pyrollo[2,3-d]pyrimidine derivatives as generically and specifically described in PCT Publication No. WO 03/013541 and U.S. Publication No. 2008/0139587.

SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors which include without limitation 2-aminopurine.

sterol biosynthesis inhibitors which include without limitation squalene epoxidase and CYP2D6.

VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

In some embodiments, the cysteine protease inhibitor such as a Cathepsin L inhibitor and/or Ctr1/Ctr2 inhibitor can be administered with the platinum-based therapeutic instead of administration of a platinum-based therapeutic alone, e.g., instead of a platinum-based therapeutic as a first line therapy or a second line therapy.

In embodiments, a hormone and/or steroid can be administered in combination with a platinum-based chemotherapeutic and a cysteine protease inhibitor such as a Cathepsin L inhibitor and/or Ctr1/Ctr2 inhibitor. Such co-administration may be particularly beneficial as hormones or steroids can elevate the level of expression of a functional Ctr1 protein. See, e.g., Hardman et al. (2006) Placenta 27:968-977. Additionally, the steroid hormonal activation of Ctr1 gene expression is also supported by a publicly available database in which the human Ctr1 gene (SLC31A1) promoter is analyzed (http://genome.ucsc.edu). Experiments have demonstrated that the Ctr1 promoter is bound by transcription factors that are known to play direct or indirect roles in steroid hormone-activated gene transcription such as androgen or progesterone. This includes experimentally validated binding sites for the following relevant transcription factors: FoxP, HNF4, FoxA, TCF7L2 and Myc.

Examples of hormones and steroids include: 17a-ethinylestradiol (Estinyl®, Ethinoral®, Feminone®, Orestralyn®), diethylstilbestrol (Acnestrol®, Cyren A®, Deladumone®, Diastyl®, Domestrol®, Estrobene®, Estrobene®, Estrosyn®, Fonatol®, Makarol®, Milestrol®, Milestrol®, Neo-Oestronol I®, Oestrogenine®, Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AV)), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In embodiments, the platinum-based chemotherapeutic and the cysteine protease inhibitor such as a Cathepsin L inhibitor and/or Ctr1/Ctr2 inhibitor may be administered in combination with an anti-microbial (e.g., leptomycin B).

In an embodiment, the platinum-based chemotherapeutic and the cysteine protease inhibitor such as a Cathepsin L inhibitor and/or Ctr1/Ctr2 inhibitor may be administered in combination with an agent or procedure to mitigate potential side effects from the agent compositions such as diarrhea, nausea and vomiting.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, lanreotide, vapreotide (Sanvar®, Sanvar IR®), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the methods described herein are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), prochlorperazine (Bayer A 173®, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some embodiments, the platinum-based chemotherapeutic and the cysteine protease inhibitor such as a Cathepsin L inhibitor and/or Ctr1/Ctr2 inhibitor may be administered in combination with an immunosuppressive agent. Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), calcineurin inhibitors (e.g., Tacrolimus (Prograf®, Protopic®), sirolimus (Rapamune®), everolimus (Afinitor®), cyclophosphamide (Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNF.alpha. antibody (e.g., Infliximab (Remicade®) or adalimumab (Humira®)).

In some embodiments, a platinum-based chemotherapeutic and the cysteine protease inhibitor such as a Cathepsin L inhibitor and/or Ctr1/Ctr2 inhibitor are administered in combination with a CYP3A4 inhibitor (e.g., ketoconazole (Nizoral®, Xolegel®), itraconazole (Sporanox®), clarithromycin (Biaxin®), atazanavir (Reyataz®), nefazodone (Serzone®, Nefadar®), saquinavir (Invirase®), telithromycin (Ketek®), ritonavir (Norvir®), amprenavir (also known as Agenerase, a prodrug version is fosamprenavir (Lexiva®, Telzir®), indinavir (Crixivan®), nelfinavir (Viracept®), delavirdine (Rescriptor®) or voriconazole (Vfend®)).

When employing the methods or compositions, other agents used in the modulation of tumor growth or metastasis in a clinical setting, such as antiemetics, can also be administered as desired.

Exemplary agents that can be administered with a platinum-based chemotherapeutic and a cysteine protease inhibitor such as a Cathepsin L inhibitor and/or Ctr1/Ctr2 inhibitor include, e.g., when the platinum-based chemotherapeutic is cisplatin: pemetrexed (ALIMTA®), vinorelbine (Navelbine®), gemcitabine (Gemzar®) vinblastine (Velban®, Velsar®), dacarbazine (DTIC-Dome®) temozolomide (Methazolastone®, Temodar®), 5FU (Adrucil®, Efudex®, Fluoroplex®), cyclophosphamide (Cytoxan®, Neosar®, Endoxan®, Procytox®, Revimmune™), bleomycin (Blenoxane®), etoposide (Toposar®, VePesid®), ifosfamide (Mitoxana®), paclitaxel(Taxol®), methotrexate (Abitrexate®, Folex®, Methotrexate®, Mexate®, Rheumatrex®, Trexall®), doxorubicin (Adriamycin®), vincristine (Vincasar®, Oncovin®), mitomycin (Mitozytrex®, Mutamycin®), docetaxel (Taxotere®), vinorelbine (Navelbine®), and combinations of the above agents. The above agents may also be administered in conjunction with surgery and/or radiation.

When the platinum-based chemotherapeutic is carboplatin, exemplary agents that can be administered with carboplatin and the cysteine protease inhibitor such as a Cathepsin L inhibitor and/or Ctr1/Ctr2 inhibitor include, e.g., irinotecan (Camptosar®), leucovorin (Wellcovorin®), 5FU (Adrucil®, Efudex®, Fluoroplex®), capecitabine (Xeloda®), bevacizumab (Avastin®), paclitaxel(Taxol®), cyclophosphamide (Cytoxan®, Neosar®, Endoxan®, Procytox®, Revimmune™), docetaxel (Taxotere®), gemcitabine (Gemzar®), etoposide (Toposar®, VePesid®), ifosfamide (Mitoxana®), vinorelbine (Navelbine®), doxorubicin (Adriamycin®), methotrexate (Abitrexate®, Folex®, Methotrexate®, Mexate®, Rheumatrex®, Trexall®), vincristine (Vincasar®, Oncovin®), and combinations of the above agents. The above agents may also be administered in conjunction with surgery and/or radiation.

When the platinum-based chemotherapeutic is oxaliplatin, exemplary agents that can be administered with carboplatin and the cysteine protease inhibitor such as a cysteine protease inhibitor such as a Cathepsin L inhibitor and/or Ctr1/Ctr2 inhibitor include, e.g., leucovorin (Wellcovorin®), and 5FU (Adrucil®, Efudex®, Fluoroplex®), and combinations of the above agents. The above agents may also be administered in conjunction with surgery and/or radiation.

When formulating the pharmaceutical compositions described herein, the clinician may utilize preferred dosages as warranted by the condition of the subject being treated. For example, in one embodiment, a platinum-based chemotherapeutic and a cysteine protease inhibitor such as a Cathepsin L inhibitor and/or Ctr1/Ctr2 inhibitor may be administered at a dosing schedule described herein, e.g., once every one, two, three, four, five or six weeks.

Also, in general, a platinum-based chemotherapeutic and a cysteine protease inhibitor such as a Cathepsin L inhibitor and/or Ctr1/Ctr2 inhibitor and an optional additional chemotherapeutic agent(s) do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The actual dosages of the compounds employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

The particular choice of additional anti-proliferative cytotoxic agent(s) or radiation will depend upon the diagnosis of the attending physicians and their judgment of the condition of the subject and the appropriate treatment protocol.

If the platinum-based chemotherapeutic, cysteine protease inhibitor such as a Cathepsin L inhibitor and/or Ctr1/Ctr2 inhibitor, and the additional chemotherapeutic agent(s) and/or radiation, are not administered simultaneously or essentially simultaneously, then the order of administration may be varied. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

### 3. METHODS OF IDENTIFYING PATIENTS AND PREDICTING RESPONSIVENESS

Also disclosed herein are methods for predicting the responsiveness of a subject having cancer to treatment with a platinum-based chemotherapeutic agent, methods for predicting the responsiveness of a patient having cancer to copper chelation therapy, as well as methods for identifying subjects as candidates for certain treatments. For example, the methods may involve identification of a subject as a candidate for treatment with a combination of a platinum-based chemotherapeutic and a cysteine protease inhibitor such as a Cathepsin L inhibitor and/or a Ctr1/Ctr2 inhibitor. Additionally, the disclosure may provide methods of identifying a subject having cancer as a candidate for treatment with a chemotherapeutic agent other than a platinum-based chemotherapeutic. The disclosure may also provide methods of identifying a subject having cancer as a candidate for copper chelation therapy. The methods involve identifying whether the subject has a polymorphism at codon position 25 of the SLC31A1 gene open reading frame encoding human Ctr1 protein.

a. SLC31A1 Polymorphisms

The methods comprise providing a nucleic acid-containing sample obtained from the subject; and detecting a Ctr1 nucleotide sequence selected from the group consisting of SEQ ID NO:1 and SEQ ID NO:2, as shown in Table 1. The presence of SEQ ID NO: 1 indicates that the subject is a responder to treatment with a platinum-based chemotherapeutic in the absence of a cysteine protease inhibitor such as a Cathepsin L inhibitor, and wherein the presence of SEQ ID NO: 2 indicates that the subject is a non-responder to treatment with a platinum-based chemotherapeutic in the absence of a cysteine protease inhibitor such as a Cathepsin L inhibitor. Additionally, the presence of SEQ ID NO: 1 may indicate that the subject is a responder to copper chelation therapy, and the presence of SEQ ID NO: 2 may indicate that the subject is a non-responder to copper chelation therapy.

TABLE 1

| Protein | Sequence | SEQ ID |
|---|---|---|
| NP_001851.1, high | MDHSHHMGMSYMDSNSTMQPS | SEQ ID |
| affinity copper | HHHPTTSASHSHGGGDSSMMM | NO: 1 |
| uptake protein 1 | MPMTFYFGFKNVELLFSGLVI | |
| [Homo sapiens] | NTAGEMAGAFVAVFLLAMFYE | |
| | GLKIARESLLRKSQVSIRYNS | |
| | MPVPGPNGTILMETHKTVGQQ | |
| | MLSFPHLLQTVLHIIQVVISY | |
| | FLMLIFMTYNGYLCIAVAAGA | |
| | GTGYFLFSWKKAVVVDITEHC | |
| | H | |
| p.Pro25A1a high | MDHSHHMGMSYMDSNSTMQPS | SEQ ID |
| affinity copper | HHHATTSASHSHGGGDSSMMM | NO: 2 |
| uptake protein 1 | MPMTFYFGFKNVELLFSGLVI | |
| [Homo sapiens] | NTAGEMAGAFVAVFLLAMFYE | |
| SNP | GLKIARESLLRKSQVSIRYNS | |
| | MPVPGPNGTILMETHKTVGQQ | |
| | MLSFPHLLQTVLHIIQVVISY | |
| | FLMLIFMTYNGYLCIAVAAGA | |
| | GTGYFLFSWKKAVVVDITEHC | |
| | H | |

The inventors have discovered that a single nucleotide polymorphism (SNP) in the SLC31A1 gene, which encodes human Ctr1, results in a change in the coding region such that proline-25 is mutated to an alanine residue. The sequence of SLC31A1 is presented in Table 1 as SEQ ID NO:1, while the sequence of the gene including the SNP is presented as SEQ ID NO:2. The polymorphism in SEQ ID NO:2 is abundantly represented in the Yoruban tribe in Nigeria, and is also abundantly represented in DNA samples from African American patients in the Duke CATHGEN database and sample collection. As illustrated in the Examples, expression of a gene including this SNP in mouse embryonic fibroblasts (MEFs) produces a Ctr1 protein that is present almost exclusively in a form in which the ectodomain has been cleaved. Furthermore, treatment with a cysteine protease inhibitor such as a Cathepsin L inhibitor significantly reduces the levels of cleaved Ctr1 and increases the levels of full-length Ctr1, and increases accumulation of cisplatin. These results indicate that patients having this polymorphism may be resistant to treatment with a platinum-based chemotherapeutic agent alone, but may respond to treatment with a platinum-based chemotherapeutic if it is administered in combination with a cysteine protease inhibitor such as a Cathepsin L inhibitor or a Ctr1/Ctr2 inhibitor.

b. Samples

The sample may comprise nucleic acid from the subject (e.g., a human). The nucleic acid may be DNA or RNA. The nucleic acid may be genomic. The sample may be used directly as obtained from the subject or following pretreatment to modify a character of the sample. Pretreatment may include extraction, concentration, inactivation of interfering components, and/or the addition of reagents.

Any cell type, tissue, or bodily fluid may be utilized to obtain a nucleic acid sample. Such cell types, tissues, and fluid may include sections of tissues such as biopsy and autopsy samples, frozen sections taken for histologic purposes, blood, plasma, serum, sputum, stool, tears, mucus, saliva, hair, and skin. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Nucleic acid purification may or may not be necessary.

c. Detection

The sample may comprise nucleic acid from the subject (e.g., a human). The nucleic acid may be DNA or RNA. The nucleic acid may be genomic. The sample may be used directly as obtained from the subject or following pretreatment to modify a character of the sample. Pretreatment may include extraction, concentration, inactivation of interfering components, and/or the addition of reagents.

Many methods are available for detecting a nucleic acid sequence in a sample from a subject, and may be used in conjunction with the herein described methods. These methods include large-scale SNP genotyping, exonuclease-resistant nucleotide detection, solution-based methods, genetic bit analyses, primer guided nucleotide incorporation, allele specific hybridization, and other techniques. Any method of detecting a marker may use a labeled oligonucleotide.
(1) Large Scale SNP Genotyping

Large scale SNP genotyping may include any of dynamic allele-specific hybridization (DASH), microplate array diagonal gel electrophoresis (MADGE), pyrosequencing, oligonucleotide-specific ligation, or various DNA “chip” technologies such as Affymetrix SNP chips. These methods may require amplification of the target genetic region. Amplification may be accomplished via polymerase chain reaction (PCR).

(2) Exonuclease-Resistant Nucleotide

Nucleotide sequences may be detected using a specialized exonuclease-resistant nucleotide, as described in U.S. Pat. No. 4,656,127, which is incorporated herein by reference. A primer complementary to the allelic sequence immediately 3′ to the polymorphic site may be permitted to hybridize to a target molecule obtained from the subject. If the polymorphic site on the target molecule contains a nucleotide that is complementary to the particular exonuclease-resistant nucleotide derivative present, then that derivative may be incorporated onto the end of the hybridized primer. Such incorporation may render the primer resistant to exonuclease, and thereby permit its detection. Since the identity of the exonuclease-resistant derivative of the sample may be known, a finding that the primer has become resistant to exonuclease reveals that the nucleotide present in the polymorphic site of the target molecule was complementary to that of the nucleotide derivative used in the reaction. This method may not require the determination of large amounts of extraneous sequence data.

(3) Solution-Based Method

A solution-based method may be used to determine the identity of a nucleotide sequence, as described in PCT Application No. WO91/02087, which is herein incorporated by reference. A primer may be employed that is complementary to allelic sequences immediately 3′ to a polymorphic site. The method may determine the identity of the nucleotide of that site using labeled dideoxynucleotide derivatives that, if complementary to the nucleotide of the polymorphic site, will become incorporated onto the terminus of the primer.

(4) Genetic Bit Analysis

Genetic bit analysis may use mixtures of labeled terminators and a primer that is complementary to the sequence 3′ to a polymorphic site. A labeled terminator may be incorporated, wherein it is determined by and complementary to, the nucleotide present in the polymorphic site of the target molecule being evaluated. The primer or the target molecule may be immobilized to a solid phase.

(5) Primer-Guided Nucleotide Incorporation

A primer-guided nucleotide incorporation procedure may be used to assay for nucleotide sequence, as described in Nyren, P. et al., Anal. Biochem. 208:171-175 (1993). Such a procedure may rely on the incorporation of labeled deoxynucleotides to discriminate between bases at a polymorphic site. In such a format, since the signal is proportional to the number of deoxynucleotides incorporated, polymorphisms that occur in runs of the same nucleotide may result in signals that are proportional to the length of the run.

(6) Allele Specific Hybridization

Allele specific hybridization may be used to detect a nucleotide sequence. This method may use a probe capable of hybridizing to a target allele. The probe may be labeled. A probe may be an oligonucleotide. The target allele may have between 3 and 50 nucleotides around the marker. The target allele may have between 5 and 50, between 10 and 40, between 15 and 40, or between 20 and 30 nucleotides around the marker. A probe may be attached to a solid phase support, e.g., a chip. Oligonucleotides may be bound to a solid support by a variety of processes, including lithography. A chip may comprise more than one allelic variant of a target region of a nucleic acid, e.g., allelic variants of two or more polymorphic regions of a gene.

(7) Other Techniques

Examples of other techniques for detecting alleles include selective oligonucleotide hybridization, selective amplification, or selective primer extension. Oligonucleotide primers may be prepared in which the known mutation or nucleotide difference is placed centrally and then hybridized to target DNA under conditions which permit hybridization if a perfect match is found. Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Allele specific amplification technology that depends on selective PCR amplification may be used in conjunction with the instant invention. Oligonucleotides used as primers for specific amplification may carry the mutation or polymorphic region of interest in the center of the molecule. Amplification may then depend on differential hybridization, as described in Gibbs et al. (1989) Nucleic Acids Res. 17:2437-2448), which is herein incorporated by reference, or at the extreme 3′ end of one primer where, under appropriate conditions, mismatch can prevent, or reduce polymerase extension.

Direct DNA sequencing, either manual sequencing or automated fluorescent sequencing may detect sequence variation. Another approach is the single-stranded conformation polymorphism assay (SSCP), as described in Orita M, et al. (1989) Proc. Natl. Acad. Sci. USA 86:2766-2770, which is incorporated herein by reference. The fragments that have shifted mobility on SSCP gels may be sequenced to determine the exact nature of the DNA sequence variation. Other approaches based on the detection of mismatches between the two complementary DNA strands include clamped denaturing gel electrophoresis (CDGE), as described in Sheffield V C, et al. (1991) Am. J. Hum. Genet. 49:699-706, which is incorporated herein by reference; heteroduplex analysis (HA), as described in White M B, et al. (1992) Genomics 12:301-306, which is incorporated herein by reference; and chemical mismatch cleavage (CMC) as described in Grompe M, et al., (1989) Proc. Natl. Acad. Sci. USA 86:5855-5892, which is herein incorporated by reference. A review of currently available methods of detecting DNA sequence variation can be found in a review by Grompe (1993), which is incorporated herein by reference. Grompe M (1993) Nature Genetics 5:111-117. Once a mutation is known, an allele specific detection approach such as allele specific oligonucleotide (ASO) hybridization can be utilized to rapidly screen large numbers of other samples for that same mutation. Such a technique can utilize probes that may be labeled with gold nanoparticles to yield a visual color result as described in Elghanian R, et al. (1997) Science 277:1078-1081, which is herein incorporated by reference.

A rapid preliminary analysis to detect polymorphisms in DNA sequences can be performed by looking at a series of Southern blots of DNA cut with one or more restriction enzymes, preferably with a large number of restriction enzymes.

d. Amplification

Any method of detection may incorporate a step of amplifying the nucleotide sequence. A nucleotide sequence may be amplified and then detected. Nucleic acid amplification techniques may include cloning, polymerase chain reaction (PCR), PCR of specific alleles (ASA), ligase chain reaction (LCR), nested polymerase chain reaction, self-sustained sequence replication, transcriptional amplification system, and Q-Beta Replicase, as described in Kwoh, D. Y. et al., 1988, Bio/Technology 6:1197, which is incorporated herein by reference.

Amplification products may be assayed by size analysis, restriction digestion followed by size analysis, detecting specific tagged oligonucleotide primers in reaction products, allele-specific oligonucleotide (ASO) hybridization, allele specific 5′ exonuclease detection, sequencing, and/or hybridization.

PCR-based detection means may include amplification of a plurality of markers simultaneously. PCR primers may be selected to generate PCR products that do not overlap in size and may be analyzed simultaneously. Alternatively, one may amplify different markers with primers that are differentially labeled. Each marker may then be differentially detected. Hybridization-based detection means may allow the differential detection of multiple PCR products in a sample.

Nucleic acid primers and/or oligonucleotides may be used in conjunction with any of the herein described methods and/or kits. The following oligonucleotides or primers may be present in the herein described kits and/or used in the herein described methods:

The following non-limiting Examples are intended to be purely illustrative, and show specific experiments that were carried out in accordance with the disclosure.

### EXAMPLES

### Example 1

### Ctr1 Ectodomain Cleavage in Ctr2^{+/+}, Ctr2^{+/-} and Ctr2^{-/-} MEFs

Mouse embryonic fibroblasts from wild type, Ctr2^{+/-}, and Ctr2^{-/-} littermates were cultured in medium supplemented with 10% fetal bovine serum and harvested at 90% confluency. Total proteins were isolated from the cells by homogenizing cells in ice cold PBS supplemented with 1% Triton-X, 0.1% SDS, and 1 mM EDTA. Cell debris was removed by centrifugation and the total amounts of soluble proteins were quantified in each sample. Equal amounts of proteins were separated on Tris/glycine gradient gel, transferred to nitrocellulose membrane and blocked with 5% non-fat milk in Tris buffered saline supplemented with 0.05% Tween (TBST) for 1 h. Membranes were incubated with anti-Ctr1 antibody (1:1000) followed by anti-rabbit-HRP coupled antibody (1:5000) and bands detected by enhanced chemiluminescent substrate. Anti-Tubulin antibody was used as loading control.

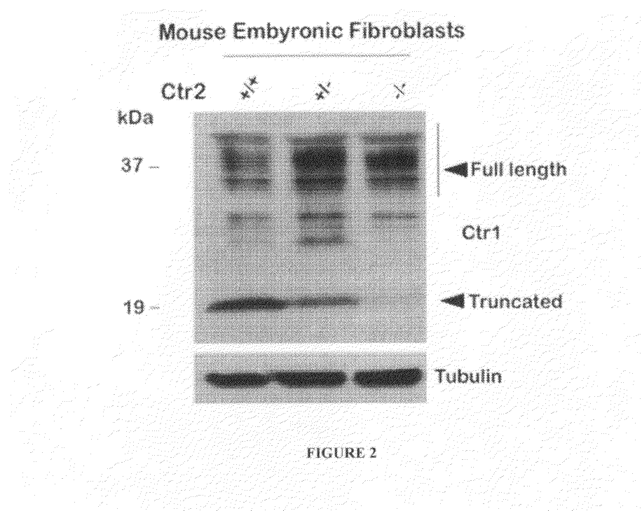

FIG. 2 illustrates immunoblotting of mouse embryonic fibroblasts (MEFs) from Wild type (Ctr2^{+/+}), heterozygous (Ctr2^{+/-}) and knock out cells (Ctr2^{-/-}) with anti-Ctr1 antibody and anti-Tubulin antibody as a control. Loss of Ctr2 results in a gene-dosage-dependent decrease in Ctr1 cleavage, as indicated by the abundance of the truncated form of Ctr1 (Truncated) versus the full length form (Full length).

### Example 2

### Cisplatin Uptake/Accumulation in Ctr2^{+/+} and Ctr2^{-/-} MEFs

Cells were treated independently with 200 μM cisplatin for 2 hours. Cisplatin and copper accumulation were measured in wild type mouse embryonic fibroblasts (Ctr2^{+/+}) and in Ctr2^{-/-} fibroblasts in four independent biological replicates. Total cell lysate was prepared, quantitated for protein concentration, digested with concentrated nitric acid and copper levels or cisplatin levels determined by inductively coupled plasma mass spectrometry (ICP-MS).

Mouse embryonic fibroblasts from wild type and Ctr2^{-/-} littermates were cultured in medium supplemented with 10% fetal bovine serum and treated with 200 μM Cisplatin for 2 hours. Cells were rinsed in three times with ice cold PBS before the cells were scraped and divided into two tubes; one for measuring metal concentration and one for protein quantification. Cell pellets from four independent cultures for each treatment groups were digested in concentrated nitric acid supplemented with 30% hydrochloric acid for 1 hour at 85° C. and mixed with ddH₂O. Copper and platinum concentrations in the digested samples were measured by inductively coupled plasma mass spectrometry (ICP-MS) and normalized to the total amount of protein in the sample.

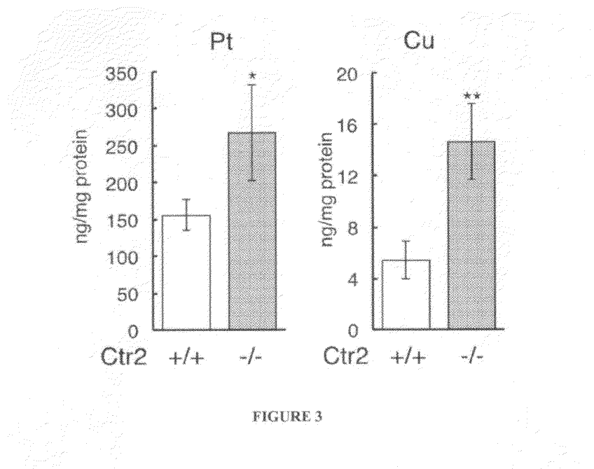

As shown in FIG. 3, a loss of Ctr2 expression leads to increased platinum and copper accumulation.

### Example 3

### Ctr1 Ectodomain Cleavage in Cathepsin L^{-/-} Cells

Mouse embryonic fibroblasts from wild type and CatL^{-/-} littermates were cultured in medium supplemented with 10% fetal bovine serum and treated with DMSO or 10M of the cell permeable cysteine protease inhibitor E64d and harvested 16 hours later. Total proteins were isolated from the cells by homogenizing cells in ice cold PBS supplemented with 1% Triton-X, 0.1% SDS, and 1 mM EDTA. Cell debris was removed by centrifugation and the total amounts of soluble proteins were quantified in each sample. Equal amounts of proteins were separated on Tris/glycine gradient gel, transferred to nitrocellulose membrane and blocked with 5% non-fat milk in Tris buffered saline supplemented with 0.05% Tween (TBST) for 1 h. Membranes were incubated with anti-Ctr1 antibody (1:1000) followed by anti-rabbit-HRP coupled antibody (1:5000) and bands detected by enhanced chemiluminescent substrate. Anti-Tubulin antibody was used as loading control.

FIG. 4 shows immunoblotting results of the analysis of protein extracts from wild type MEFs (lanes 1-4) and Cathepsin L knock out fibroblasts (Cathepsin L^{-/-}, lanes 5-8) with anti-Ctr1 antibody and anti-Tubulin antibody as a loading control. Loss of Cathepsin L (lanes 5-8) results in a dramatic reduction in the levels of cleaved Ctr1. Treatment of wild type cells with E64d (10 μM), a Cathepsin L inhibitor, also results in a dramatic reduction in the levels of cleaved Ctr1 in wild type cells (lanes 4 and 5 are duplicate biological experiments) but not Cathepsin L knock out cells (lanes 7 and 8 are duplicate biological experiments). Lanes 1 and 2 are duplicate samples from untreated wild type cells and lanes 5 and 6 are duplicate samples from untreated Cathepsin L knock out cells. Notably, as shown in FIG. 4, there appears to be an increase full-length Ctr1 in Cathepsin L^{-/-} cells when treated with the Cathepsin L inhibitor E64d (lanes 7 and 8), compared to in Cathepsin L^{-/-} cells that are not treated with E64d (lanes 5 and 6). Therefore, E64d may not only be inhibiting Cathepsin L, but may also be inhibiting another protease that may also be involved in Ctr1 ectodomain cleavage.

### Example 4

### Ctr1^{P25A} Ectodomain Cleavage

Mouse embryonic fibroblasts from Ctr1^{-/-} embryos were stably transfected with the human Ctr1 and the human Ctr1 containing the proline to alanine mutation at position 25 (Ctr1^{P25A}). Cells were cultured in medium supplemented with 20% fetal bovine serum and treated with DMSO or 10 μM cysteine protease Cathepsin L, Z-FY(t-Bu)-DMK and harvested 16 hours later. Total proteins were isolated from the cells by homogenizing cells in ice cold PBS supplemented with 1% Triton-X, 0.1% SDS, and 1 mM EDTA. Cell debris was removed by centrifugation and the total amounts of soluble proteins were quantified in each sample. Equal amounts of proteins were separated on tris/glycine gradient gel, transferred to nitrocellulose membrane and blocked with 5% non-fat milk in Tris buffered saline supplemented with 0.05% Tween (TBST) for 1 h. Membranes were incubated with anti-Ctr1 antibody (1:1000) followed by anti-rabbit-HRP coupled antibody (1:5000) and bands detected by enhanced chemiluminescent substrate. Anti-Actin antibody was used as loading control.

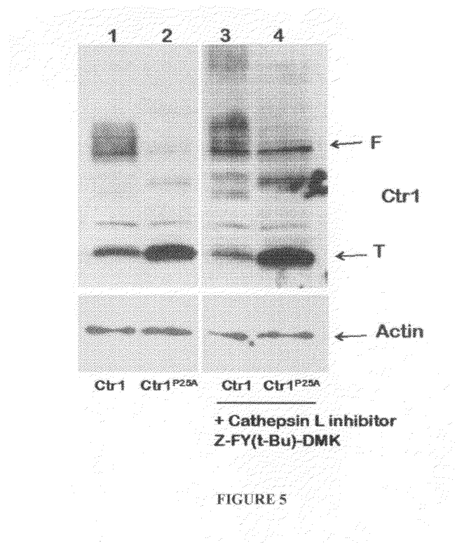

As shown in FIG. 5, cells expressing the Ctr1 proline 25 to alanine mutant protein (Ctr1^{P25A}) show a dramatic reduction in full length form that can be alleviated by treatment with Cathepsin L inhibitors. This figure shows immunoblotting results of the analysis of protein extracts from wild type mouse cells and cells expressing the Ctr1 proline 25 to alanine mutant protein (Ctr1^{P25A}) with anti-Ctr1 antibody and anti-Actin antibody as a loading control. Inhibition of the cysteine protease Cathepsin L, Z-FY(t-Bu)-DMK (10 μM) (lanes 3 and 4) results in an increased levels of the copper and cisplatin binding full length form of Ctr1. This immunoblot results are from the same membrane, same x-ray film, and exposed the same time.

### Example 5

### Cisplatin Uptake/Accumulation in Ctr1^{-/-} and Ctr1^{P25A} MEFs

Mouse embryonic fibroblasts from Ctr1^{-/-} embryos were stably transfected with the human Ctr1 and the human Ctr1 containing the proline to alanine mutation at position 25 (Ctr1^{P25A}). Cells were cultured in medium supplemented with 20% fetal bovine serum and treated with DMSO or 10 μM cysteine protease Cathepsin L, Z-FY(t-Bu)-DMK over night and then PBS or 200 μM Cisplatin were added to the cells for 2 hours accumulation. Cells were rinsed in three times with ice cold PBS before the cells were scraped and divided into two tubes; one for measuring metal concentration and one for protein quantification. Cell pellets from four independent cultures for each treatment groups were digested in concentrated nitric acid supplemented with 30% hydrochloric acid for 1 hour at 85° C. and mixed with ddH₂O. Platinum concentrations in the digested samples were measured by inductively coupled plasma mass spectrometry (TCP-MS) and normalized to the total amount of protein in the sample.

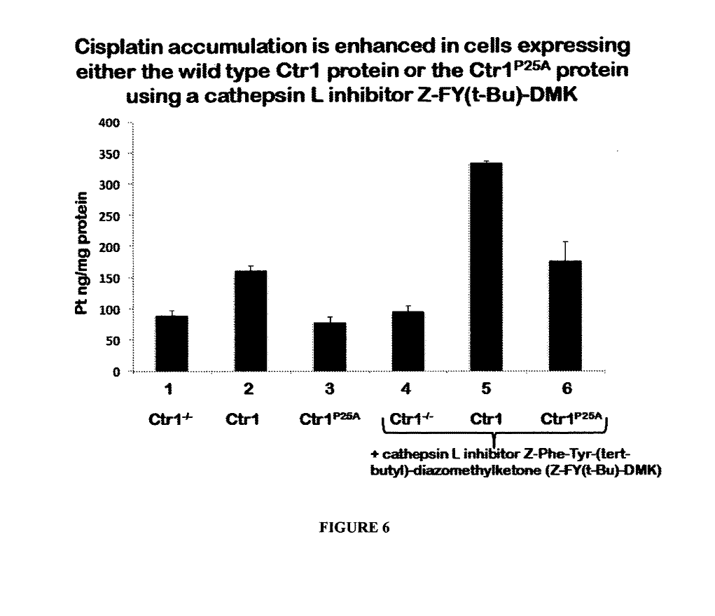

As shown in FIG. 6, cisplatin accumulation carried out by wild type Ctr1, or cells expressing the Ctr1 proline 25 to alanine mutant protein (Ctr1^{P25A}), is enhanced by co-treatment of cells with cisplatin and the Cathepsin L inhibitor Z-FY(t-Bu)-DMK. Mouse Ctr1^{-/-} MEFs were transfected with vector alone (Ctr1^{-/-}), the expression vector expressing wild type Ctr1 (Ctr1) or the Ctr1^{P25A} mutant protein (Ctr1^{P25A}). Cells were incubated with cisplatin (200 μM for 2 h), or cisplatin plus the Cathepsin L inhibitor Z-FY(t-Bu)-DMK (10 μM) as indicated, harvested and cisplatin accumulation measured by ICP-MS and plotted on the Y-axis.

### Example 6

### Experiments in Cancer Cell Lines

Experiments planned to further analyze the relationship between the inhibition of Ctr1 ecto-domain cleavage and enhancing the efficacy of platinum-based drug uptake will include, but are not limited to: 1) the evaluation of cisplatin uptake and sensitivity in cancer cell lines in the presence and absence of cysteine protease inhibitors. These cell lines will include, but are not limited to ovarian cancer (OVCA420 cells), breast cancer (such as MCF7 cells), testicular cancer and other cancer cell lines.

All patents, publications and references cited herein are hereby fully incorporated by reference. In case of conflict between the present disclosure and incorporated patents, publications and references, the present disclosure should control.

1. A method of reducing the proliferation of a cancer cell, comprising contacting the cancer cell with a platinum-based chemotherapeutic and a cysteine protease inhibitor.

2. The method of claim 1, wherein the cancer cell is selected from the group consisting of a head cancer, neck cancer, ovarian cancer, breast cancer, pancreatic cancer, testicular cancer, melanoma, bladder cancer, lung cancer, sarcoma, squamous cell carcinoma, or small cell lung cancer cell.

3. The method of claim 1, wherein the platinum-based chemotherapeutic is selected from the group consisting of cisplatin, oxaliplatin, carboplatin, satraplatin and picoplatin.

4. The method of claim 3, wherein the platinum-based chemotherapeutic is cisplatin.

5. The method of claim 1, wherein the cysteine protease inhibitor is a cathepsin inhibitor.

6. The method of claim 5, wherein the cathepsin inhibitor is an inhibitor of at least one of Cathepsin L, B, C, F, H, K, V, O, S and W.

7. The method of claim 6, wherein the cathepsin inhibitor is an inhibitor of at least one of Cathepsin L, B, and H.

8. The method of claim 7, wherein the cathepsin inhibitor is a Cathepsin L inhibitor.

9. The method of claim 8, wherein the Cathepsin L inhibitor is selected from the group consisting of Z-FY(tBu)-DMK and E64d.

10. The method of claim 1, wherein the cancer cell is contacted with the platinum-based chemotherapeutic and the cysteine protease inhibitor in vitro, in vivo or ex vivo.

11. The method of claim 1, wherein the cancer cell is first contacted with the cysteine protease inhibitor, and subsequently contacted with the platinum-based chemotherapeutic.

12. The method of claim 1, wherein the cancer cell is first contacted with the platinum-based chemotherapeutic, and subsequently contacted with the cysteine protease inhibitor.

13. The method of claim 1, wherein the cancer cell is simultaneously contacted with the cysteine protease inhibitor and the platinum-based chemotherapeutic.

14. The method of claim 1, further comprising contacting the cell with an additional chemotherapeutic agent.

15. The method of claim 1, further comprising contacting the cell with a hormone and/or a steroid.

16. A method of treating cancer in a subject in need of treatment, comprising administering to the subject a platinum-based chemotherapeutic and a cysteine protease inhibitor, in amounts effective to treat the cancer.

17. The method of claim 16, wherein the cancer is selected from the group consisting of head cancer, neck cancer, ovarian cancer, breast cancer, pancreatic cancer, testicular cancer, melanoma, bladder cancer, lung cancer, sarcoma, squamous cell carcinoma, or small cell lung cancer.

18. The method of claim 16, wherein the platinum-based chemotherapeutic is selected from the group consisting of cisplatin, oxaliplatin, carboplatin, satraplatin and picoplatin.

19. The method of claim 18, wherein the platinum-based chemotherapeutic is cisplatin.

20. The method of claim 16, wherein the cysteine protease inhibitor is a cathepsin inhibitor.

21. The method of claim 20, wherein the cathepsin inhibitor is an inhibitor of at least one of Cathepsin L, B, C, F, H, K, V, O, S and W.

22. The method of claim 21, wherein the cathepsin inhibitor is an inhibitor of at least one of Cathepsin L, B, and H.

23. The method of claim 22, wherein the cathepsin inhibitor is a Cathepsin L inhibitor.

24. The method of claim 23, wherein the Cathepsin L inhibitor is selected from the group consisting of Z-FY(tBu)-DMK and E64d.

25. The method of claim 16, wherein the platinum-based chemotherapeutic and the cysteine protease inhibitor are each independently administered parenterally or orally.

26. The method of claim 16, wherein the cysteine protease inhibitor is administered to the subject first, followed by subsequent administration of the platinum-based chemotherapeutic.

27. The method of claim 16, wherein the cysteine protease inhibitor and the platinum-based chemotherapeutic are administered simultaneously to the subject.

28. The method of claim 16, further comprising administering an additional chemotherapeutic agent to the subject.

29. The method of claim 16, further comprising administering a hormone and/or a steroid to the subject.

30. A method of predicting responsiveness of a subject having cancer to treatment with a platinum-based chemotherapeutic agent, comprising:
providing a nucleic acid-containing sample obtained from the subject; and
detecting a Ctr1 nucleotide sequence selected from the group consisting of SEQ ID NO:1 and SEQ ID NO:2;
wherein the presence of SEQ ID NO: 1 indicates that the subject is a responder to treatment with a platinum-based chemotherapeutic in the absence of a cysteine protease inhibitor, and wherein the presence of SEQ ID NO:2 indicates that the subject is a non-responder to treatment with a platinum-based chemotherapeutic in the absence of a cysteine protease inhibitor.

31. The method of claim 30, wherein the nucleic acid-containing sample is a nucleic acid extract from a biological sample from the subject.

32. The method of claim 31, wherein the biological sample comprises blood, saliva or buccal cells.

33. The method of claim 31, further comprising preparing the nucleic acid extract from the biological sample prior to the detecting step.

34. The method of claim 31, further comprising obtaining the biological sample from the subject prior to the preparing step.

35. The method of claim 30, wherein the detection step comprises:
a. amplifying a nucleic acid comprising the Ctr1 nucleotide sequence; and
b. detecting the amplified nucleic acids, thereby detecting the sequence.

36. The method of claim 35, wherein the Ctr1 nucleotide sequence is detected by sequencing.

37. The method of claim 30, wherein the subject is a human.

38. The method of claim 30, wherein the subject has a cancer selected from the group consisting of a head cancer, neck cancer, ovarian cancer, breast cancer, pancreatic cancer, testicular cancer, melanoma, bladder cancer, lung cancer, sarcoma, squamous cell carcinoma, or small cell lung cancer.

39. The method of claim 30, wherein the platinum-based chemotherapeutic is selected from the group consisting of cisplatin, oxaliplatin, carboplatin, satraplatin and picoplatin.

40. The method of claim 39, wherein the platinum-based chemotherapeutic is cisplatin.

41. The method of claim 30, wherein the cysteine protease inhibitor is a cathepsin inhibitor.

42. The method of claim 41, wherein the cathepsin inhibitor is an inhibitor of at least one of Cathepsin L, B, C, F, H, K, V, O, S and W.

43. The method of claim 42, wherein the cathepsin inhibitor is an inhibitor of at least one of Cathepsin L, B, and H.

44. The method of claim 43, wherein the cathepsin inhibitor is a Cathepsin L inhibitor.

45. The method of claim 44, wherein the Cathepsin L inhibitor is selected from the group consisting of Z-FY(tBu)-DMK and E64d.

46. A method of treating cancer in a subject in need of treatment, comprising:
providing a nucleic acid-containing sample obtained from the subject;
detecting a Ctr1 nucleotide sequence selected from the group consisting of SEQ ID NO:1 and SEQ ID NO:2; and
if the detecting step detects the presence of SEQ ID NO:2, the method further comprises administering to the subject a therapeutically effective amount of a platinum-based chemotherapeutic and a cysteine protease inhibitor.

47. The method of claim 46, wherein the nucleic acid-containing sample is a nucleic acid extract from a biological sample from the subject.

48. The method of claim 47, wherein the biological sample comprises blood, saliva or buccal cells.

49. The method of claim 47, further comprising preparing the nucleic acid extract from the biological sample prior to the detecting step.

50. The method of claim 47, further comprising obtaining the biological sample from the subject prior to the preparing step.

51. The method of claim 46, wherein the detection step comprises:
a. amplifying a nucleic acid comprising the Ctr1 nucleotide sequence; and
b. detecting the amplified nucleic acids, thereby detecting the sequence.

52. The method of claim 51, wherein the Ctr1 nucleotide sequence is detected by sequencing.

53. The method of claim 51, wherein the amplified nucleic acids are detected by hybridizing an oligonucleotide probe to the

54. The method of claim 46, wherein the subject is a human.

55. The method of claim 46, wherein the subject has a cancer selected from the group consisting of a head cancer, neck cancer, ovarian cancer, breast cancer, pancreatic cancer, testicular cancer, melanoma, bladder cancer, lung cancer, sarcoma, squamous cell carcinoma, or small cell lung cancer.

56. The method of claim 46, wherein the platinum-based chemotherapeutic is selected from the group consisting of cisplatin, oxaliplatin, carboplatin, satraplatin and picoplatin.

57. The method of claim 56, wherein the platinum-based chemotherapeutic is cisplatin.

58. The method of claim 46, wherein the cysteine protease inhibitor is a cathepsin inhibitor.

59. The method of claim 58, wherein the cathepsin inhibitor is an inhibitor of at least one of Cathepsin L, B, C, F, H, K, V, O, S and W.

60. The method of claim 59, wherein the cathepsin inhibitor is an inhibitor of at least one of Cathepsin L, B, and H.

61. The method of claim 60, wherein the cathepsin inhibitor is a Cathepsin L inhibitor.

62. The method of claim 61, wherein the Cathepsin L inhibitor is selected from the group consisting of Z-FY(tBu)-DMK and E64d.

63. The method of claim 46, wherein the cysteine protease inhibitor is administered to the subject first, followed by subsequent administration of the platinum-based chemotherapeutic.

64. The method of claim 46, wherein the cysteine protease inhibitor and the platinum-based chemotherapeutic are administered simultaneously to the subject.

65. The method of claim 46, further comprising administering an additional chemotherapeutic agent to the subject.

66. The method of claim 46, further comprising administering a hormone and/or a steroid to the subject.

67. A method of predicting responsiveness of a subject having cancer to copper chelation therapy, comprising:
providing a nucleic acid-containing sample obtained from the subject; and
detecting a Ctr1 nucleotide sequence selected from the group consisting of SEQ ID NO:1 and SEQ ID NO:2;
wherein the presence of SEQ ID NO:1 indicates that the subject is a responder to copper chelation therapy, and wherein the presence of SEQ ID NO:2 indicates that the subject is a non-responder to copper chelation therapy.


SEQUENCE LISTING (2 sequences)

SEQ ID NO: 1 - protein, 190 aa, Homo sapien
MDHSHHMGMSYMDSNSTMQPSHHHPTTSASHSHGGGDSSMMMMPMTFYFGFKNVELLFSGLVINTAGEMAGAFVAVFLLAMFYEGLKIARESLLRKSQVSIRYNSMPVPGPNGTILMETHKTVGQQMLSFPHLLQTVLHIIQVVISYFLMLIFMTYNGYLCIAVAAGAGTGYFLFSWKKAVVVDITEHCH

SEQ ID NO: 2 - protein, 190 aa, Homo sapien
MDHSHHMGMSYMDSNSTMQPSHHHATTSASHSHGGGDSSMMMMPMTFYFGFKNVELLFSGLVINTAGEMAGAFVAVFLLAMFYEGLKIARESLLRKSQVSIRYNSMPVPGPNGTILMETHKTVGQQMLSFPHLLQTVLHIIQVVISYFLMLIFMTYNGYLCIAVAAGAGTGYFLFSWKKAVVVDITEHCH